(19) United States
(12) Patent Application Publication
(10) Pub. No.: US 2009/0156525 A1
(43) Pub. Date: Jun. 18, 2009

(54) Low-Density Lipoprotein Receptor-Related Protein 2 Clears Amyloid-Beta Peptide A cross the Blood-Brain Barrier via Apolipoprotein J

(76) Inventors: Berislav V. Zlokovic, Rochester, NY (US); Rashid Deane, Rochester, NY (US)

Correspondence Address: NIXON & VANDERHYE, PC, 901 NORTH GLEBE ROAD, 11TH FLOOR, ARLINGTON, VA, 22203, US

(21) Appl. No.: 12/085,029

(22) PCT Filed: Nov. 15, 2006
(86) PCT No.: PCT/US2006/044401

§ 371 Date: Dec. 24, 2008

Related U.S. Application Data
(60) Provisional application No. 60/736,327, filed on Nov. 15, 2005.

(51) Int. Cl. A61K 31/7088; G01N 33/53; A61P 25/28

(52) U.S. Cl. 514/44; 435/7.1

(57) ABSTRACT

Low-density lipoprotein receptor-related protein 2 (LRP2) is a potential receptor to regulate the level of apolipoprotein J (apoJ) in the central nervous system, which is the major carrier of amyloid-β peptide (Aβ). ApoJ is cleared from brain interstitial fluid (ISF) and cerebrospinal fluid (CSF) by LRP2-mediated transcytosis across epithelial and endothelial barriers. At physiological ISF/CSF levels, apoJ is rapidly transported by LRP2 across the blood-brain barrier (BBB). Importantly, apoJ also substantially enhances clearance from the brain of amyloidogenic Aβ isoforms (i.e., higher β-sheet content such as Aβ42) as apoJ-Aβ by LRP2-mediated transport.

### CROSS-REFERENCE TO RELATED APPLICATIONS

This application claims the benefit of provisional U.S. Application No. 60/736,327, filed Nov. 15, 2005.

### FEDERALLY-SPONSORED RESEARCH OR DEVELOPMENT

The U.S. Government has certain rights in this invention as provided for by the terms of NIH grant AG023084 from the Department of Health and Human Services.

### BACKGROUND OF THE INVENTION

This invention relates to Alzheimer's disease (AD) and its etiology: the low-density lipoprotein receptor-related protein 2 (LRP2) and its ligand, apolipoprotein J (apoJ), mediates clearance of amyloid-β peptide (Aβ) from the central nervous system (CNS). Products and processes used therein are provided.

ApoJ, an 80 kDa sulfated glycoprotein, is expressed in neural and non-neural tissues (Jenne & Tschopp, 1992; Danik et al, 1993). It is involved in inhibition of complement-mediated cytolysis, cell aggregation and chemotaxis, cell death/apoptosis and lipoprotein binding. ApoJ is a major carrier protein for Aβ in plasma and cerebrospinal fluid (CSF) (Ghiso et al., 1993; Koudinov et al., 1994). It is a component of senile plaque in Alzheimer's disease (AD) and Down's syndrome (McGeer et al., 1992; Choi-Miura et a/, 1992). In AD, hippocampal apoJ mRNA is increased (May & Finch, 1992; May et al., 1989).

Astrocytes secrete apoJ in lipoprotein particles that do not contain apoE (Fagan et al., 1999). Lipidated apoJ protects Aβ from proteolytic degradation and prevents its aggregation in vitro (Matsubara et a/, 1996). ApoJ increases Aβ neurotoxicity (Oda et al., 1995; Boggs et al., 1996; Lambert et al., 1998). In a mouse model of neurodegeneration, apoJ expression is increased in astrocytes (Pasinetti et al., 1993). Deletion of the apoJ gene in PDAPP mice, a transgenic mouse model that develops AD-like neuropathology, results in less dystrophic neurites, reduced fibrillar deposits, and increased soluble Aβ in brain (DeMattos et al., 2002).

LRP2 (also called megalin or glycoprotein 330) is a receptor for apoJ, and mediates its endocytosis and lysosomal degradation (Kounnas et al., 1995) and transcytosis across the blood-brain barrier (BBB) and blood-CSF barrier (Zlokovic et al., 1996). LRP2 is expressed on the choroid plexus, cerebral endothelium (BBB), ventricular ependyma (Zlokovic et al., 1996; Chun et al., 1999), and neurons and astrocytes (Rebeck & Hyman, 1999). LRP2 at the BBB is saturated at physiological plasma levels of apoJ which precludes influx of Aβ into brain via apoJ/LRP2 route (Zlokovic et al., 1996). In contrast, LRP1 (low-density lipoprotein receptor-related protein 1) plays a key role in Aβ clearance from brain, across the BBB, in an isoform specific manner, i.e., the more amyloidogenic longer Aβ isoforms (higher β-sheet content) are inefficiently cleared across the BBB (Deane et al., 2004).

Since the role of LRP2 in controlling apoJ levels in brain and clearance of Aβ from brain interstitial fluid (ISF) and CSF was unclear, we addressed this issue by using a brain clearance technique that we developed. The results of this study can be used to improve the understanding of the pathogenesis of Alzheimer's disease and mechanisms of disease. New and nonobvious modes of diagnosis and treatment are suggested by this discovery. Other advantages of the invention are discussed below or would be apparent to persons in the art from the disclosure herein.

### SUMMARY OF THE INVENTION

The invention may be used to diagnose symptomatic and asymptomatic subjects, to identify those at risk for disease or already affected thereby, to determine the stage of disease or its progression, to intervene earlier in or alter the disease's natural history, to provide a target for therapeutic or preventative treatments, to screen drugs or compare medical regimens, to determine the effectiveness of a drug or medical regimen, or any combination thereof. Reduced low-density lipoprotein receptor-related protein 2 (LRP2) (e.g., decreased level of expression or clearance of apolipoprotein J) may be indicative of Alzheimer's disease (AD) or risk of development thereof. Enhanced LRP2 (e.g., ectopic expression, increased expression or clearance of apolipoprotein J) may be used to treat AD or to reduce the risk of development thereof.

In one embodiment of the invention, reagents are provided in kit form that can be used for performing the methods such as the following: diagnosis, identification of those at risk for disease or already affected, or determination of the stage of disease or its progression. In addition, the reagents may be used in methods related to the treatment of disease such as the following: evaluation whether or not it is desirable to intervene in the disease's natural history, alteration of the course of disease, early intervention to halt or slow progression, promotion of recovery or maintenance of function, provision of targets for beneficial therapy or prevention, comparison of candidate drugs or medical regimens, or determination of the effectiveness of a drug or medical regimen. Instructions for performing these methods, reference values and positive/negative controls, and relational databases containing patient information (e.g., genotype, medical history, symptoms, transcription or translation yields from gene expression, physiological or pathological findings) are other products that can be considered aspects of the invention.

In other embodiments of the invention, methods for diagnosis and treatment (e.g., therapy of existing disease or prevention of disease in subject at risk for developing disease) are provided. For screening of drugs and clinical trials, the respective drug and medical regimen selected are also considered embodiments of the invention. The amount and extent of treatment administered to a cell, tissue, or subject in need of therapy or prevention is effective in treating the affected cell, tissue, or subject. One or more properties/functions of affected endothelium or cells thereof, or the number/severity of symptoms of an affected subject, may be improved, reduced, normalized, ameliorated, or otherwise successfully treated. The invention may be used alone or in combination with other known methods. Instructions for performing these methods, reference values and positive/negative controls, and relational databases containing patient information are considered further aspects of the invention. The subject may be any animal or human. Mammals, especially humans and rodent or primate models of disease, may be treated. Thus, both veterinary and medical methods are contemplated.

Further aspects of the invention will be apparent to a person skilled in the art from the following description of specific embodiments and the claims, and generalizations thereto.

### BRIEF DESCRIPTION OF THE DRAWING

FIG. 1 shows the effects on Aβ42 and ApoJ clearance from brain and cerebrospinal fluid (CSF). (A) ¹²⁵I-apoJ (40 nM, TCA-precipitable) clearance in the absence and presence of RAP (5 μM), anti-LRP2 (Rb 6286, 60 μg/ml, from Dr. S. Argraves), and anti-LRP1 (N20, 60 μg/ml). (B) Time-dependent clearance of ¹²⁵I-apoJ and ¹²⁵I-Aβ42 at 40 nM vs. ¹⁴C-inulin from brain ISF. Each point represents a single experiment. (C) Time-appearance into CSF (top) and plasma (bottom) of ¹²⁵I-apoJ and ¹⁴C-inulin injected into brain interstitial fluid (ISF) (D) Effects of apoJ on ¹²⁵I-Aβ42 clearance at the BBB and effect of anti-LRP2 (Rb 6286, 60 μg/ml). (E) CSF clearance of ¹²⁵I-apoJ (40 nM) from the lateral ventricle. Mean±s.e.m., n=3-5.

### DESCRIPTION OF SPECIFIC EMBODIMENTS OF THE INVENTION

While apolipoprotein J (apoJ) is a major carrier of Aβ in biological fluids including plasma, brain interstitial fluid (ISF), and cerebrospinal fluid (CSF), its role in Aβ clearance from the central nervous system (CNS) is poorly understood. The low density lipoprotein receptor 2 (LRP2), a member of the LDL receptor family and the receptor for apoJ, is an endocytotic receptor which also participates in transcytosis of its ligands across the epithelial and endothelial barriers. LRP2 is expressed on brain endothelial and epithelial cells at the critical regulatory transport sites in the central nervous system (CNS) between the blood and brain including the blood-brain barrier (BBB) and the choroid plexus epithelium of the blood-CSF barrier. Thus, LRP2 is a potential receptor to regulate the level of apoJ in the brain, as well as of apoJ-associated ligands including Aβ by controlling their transport between brain and blood. Here, we used our clearance technique from the brain and CSF to investigate the role of LRP2 in apoJ clearance and elimination of Aβ from the CNS in the form of apoJ-Aβcomplexes. By using a panel of specific antibodies against the LDL receptor family members (e.g., LDLR, VLDLR, LRP1, LRP2) and the receptor associated protein (RAP) which blocks both LRP1 and LRP2, we showed that apoJ at physiological ISF/CSF levels is rapidly transported across the BBB and from CSF via LRP2. Importantly, our data suggest that apoJ also substantially enhances the clearance from brain of high β-sheet content ¹²⁵I-Aβ42 via LRP2. Therefore, LRP2, the receptor for apoJ, could play a key role in regulating apoJ levels in the CNS and clearance of amyloidogenic Aβ isoforms (i.e., higher β-sheet content) from brain ISF. A subject with Alzheimer's disease or at risk for developing Alzheimer's disease may be treated by increasing LRP2-mediated removal of amyloid-β peptide (Aβ) at the blood-brain barrier in the subject in the direction from brain to blood. This may be accomplished by an expression construct encoding LRP2 or a functional domain thereof (i.e., binding and transporting a complex of apoJ-Aβ) to increase expression of LRP2 or its activity.

Preparations of endothelial cells, isolated tissues, and in vitro cell cultures may be provided from brain (e.g., microvasculature) or other organs (e.g., skin) of subjects at risk for Alzheimer's disease, affected by the disease, or not. In particular, tissues like endothelium, smooth muscle, blood vessels and capillaries of the brain, temporal and leptomeningeal arteries, or any other tissues that express LRP2 can be examined. Blood and bone marrow cells might also be used. They can be obtained as biopsy or autopsy material; cells of interest may be isolated therefrom and then cultured. Also provided are extracts of cells; at least partially purified DNA, RNA, and protein therefrom; and methods for their isolation. These reagents can be used to establish detection limits for assays, absolute amounts of gene expression that are indicative of disease or not, ratios of gene expression that are indicative of disease or not, and the significance of differences in such values. These values for positive and/or negative controls can be measured at the time of assay, before an assay, after an assay, or any combination thereof. Values may be recorded on storage medium and manipulated with computer software; storage in a database allows retrospective or prospective study. Gene expression (e.g., detected at the level of chromatin, transcript, or protein) and protein activity of LRP2 (e.g., clearance of apoJ-Aβ complexes from the brain) may be decreased in a subject affected by Alzheimer's disease or at risk of developing Alzheimer's disease.

Polynucleotides representative of genes whose expression is decreased in Alzheimer's disease may be used to identify, isolate, or detect complementary polynucleotides by binding assays. Similarly, polypeptides representative of the gene products that are decreased in Alzheimer's disease may be used to identify, isolate, or detect interacting proteins by binding assays. Optionally, bound complexes including interacting proteins may be identified, isolated, or detected indirectly though a specific binding molecule (e.g., antibody) for the gene product that is decreased in Alzheimer's disease. For the receptor-ligand system studied here, LRP2 and apoJ are interacting proteins. Candidate compounds to treat Alzheimers disease may interact with at least one gene, transcript, or protein which is a component of the receptor-ligand system to increase receptor activity (i.e., vascular clearance of Aβ), and be screened for their ability to provide therapy or prevention. These products may be used in assays (e.g., diagnostic methods) or for treatment; conveniently, they are packaged as assay kits or in pharmaceutical form.

### Assaying Polynucleotides or Polypeptides

Binding of polynucleotides or polypeptides may take place in solution or on a substrate. The assay format may or may not require separation of bound from not bound. Detectable signals may be direct or indirect, attached to any part of a bound complex, measured competitively, amplified, or any combination thereof. A blocking or washing step may be interposed to improve sensitivity and/or specificity. Attachment of a polynucleotide or polypeptide, interacting protein (e.g., RAP), or specific binding molecule to a substrate before, after, or during binding results in capture of an unattached species. See U.S. Pat. Nos. 5,143,854 and 5,412,087. Abundance may be measured at the level of protein and/or transcripts of a component of the receptor-ligand system.

The polynucleotide, polypeptide, or specific binding molecule may be attached to a substrate. The substrate may be solid or porous and it may be Formed as a sheet, bead, or fiber. The substrate may be made of cotton, silk, or wool; cellulose, nitrocellulose, uncharged nylon, or positively-charged nylon; natural rubber, butyl rubber, silicone rubber, or styrene-butadiene rubber; agarose or polyacrylamide; crystalline, amorphous, or impure silica (e.g., quartz) or silicate (e.g., glass); polyacrylonitrile, polycarbonate, polyethylene, polymethyl methacrylate, polymethylpentene, polypropylene, polystyrene, polysulfone, polytetrafluoroethylene, polyvinylidenefluoride, polyvinyl acetate, polyvinyl chloride, or polyvinyl pyrrolidone; or combinations thereof. Optically-transparent materials are preferred so that binding can be monitored and signal transmitted by light.

Such reagents would allow capture of a molecule in solution by specific interaction between the cognate molecules and then could immobilize the molecule on the substrate. Monitoring gene expression is facilitated by using an array.

Polynucleotide, polypeptide, or specific binding molecule may be synthesized in situ by solid-phase chemistry or photolithography to directly attach the nucleotides or amino acids to the substrate. Attachment of the polynucleotide, polypeptide, or specific binding molecule to the substrate may be through a reactive group as, for example, a carboxy, amino, or hydroxy radical; attachment may also be accomplished after contact printing, spotting with a pin, pipetting with a pen, or spraying with a nozzle directly onto a substrate. Alternatively, the polynucleotide, polypeptide, or specific binding molecule may be reversibly attached to the substrate by interaction of a specific binding pair (e.g., antibody-digoxygenin/hapten/peptide, biotin-avidin streptavidin, glutathione S transferase-glutathione, maltose binding protein-maltose, polyhistidine-nickel, protein A or G/immunoglobulin); crosslinking may be used if irreversible attachment is desired.

Changes in gene expression may be manifested in the cell by affecting transcriptional initiation, transcript stability, translation of transcript into protein product, protein stability, or a combination thereof. The abundance of transcript or polypeptide can be measured by techniques such as in vitro transcription, in vitro translation, Northern hybridization, nucleic acid hybridization, reverse transcription-polymerase chain reaction (RT-PCR), run-on transcription, Southern hybridization, cell surface protein labeling, metabolic protein labeling, antibody binding, immunoprecipitation (IP), enzyme linked immunosorbent assay (ELISA), radioimmunoassay (RIA), fluorescent or histochemical staining, microscopy and digital image analysis, and fluorescence activated cell analysis or sorting (FACS).

A reporter or selectable marker gene whose protein product is easily assayed may be used for convenient detection. Reporter genes include, for example, alkaline phosphatase, β-galactosidase (LacZ), chloramphenicol acetyltransferase (CAT), β-glucuronidase (GUS), bacterial/insect/marine invertebrate luciferases (LUC), green and red fluorescent proteins (GFP and RFP, respectively), horseradish peroxidase (HRP), β-lactamase, and derivatives thereof (e.g., blue EBFP, cyan ECFP, yellow-green EYFP, destabilized GFP variants, stabilized GFP variants, or fusion variants sold as LIVING COLORS fluorescent proteins by Clontech). Reporter genes would use cognate substrates that are preferably assayed by a chromogen, fluorescent, or luminescent signal. Alternatively, assay product may be tagged with a heterologous epitope (e.g., FLAG, MYC, SV40 T antigen, glutathione transferase, hexahistidine, maltose binding protein) for which cognate antibodies or affinity resins are available.

A polynucleotide may be ligated to a linker oligonucleotide or conjugated to one member of a specific binding pair (e.g., antibody-digoxygenin/hapten/peptide epitope, biotin-avidin/streptavidin, glutathione transferase or GST-glutathione, maltose binding protein-maltose, polyhistidine-nickel, protein A/G-immunoglobulin). The polynucleotide may be conjugated by ligation of a nucleotide sequence encoding the binding member. A polypeptide may be joined to one member of the specific binding pair by producing the fusion encoded such a ligated or conjugated polynucleotide or, alternatively, by direct chemical linkage to a reactive moiety on the binding member by chemical crosslinking. Such polynucleotides and polypeptides may be used as an affinity reagent to identify, to isolate, and to detect interactions that involve specific binding of a transcript or protein product of the expression construct. Before or after affinity binding of the transcript or protein product, the member attached to the polynucleotide or polypeptide may be bound to its cognate binding member. This can produce a complex in solution or immobilized to a support.

An expression construct is a recombinant polynucleotide. Its chemical form may be a deoxyribonucleic acid (DNA) and/or a ribonucleic acid (RNA); its physical form may vary in strandedness (e.g., single-stranded or double-stranded) and topology (e.g., linear or circular). The expression construct is preferably a double-stranded deoxyribonucleic acid (dsDNA) or is converted into a dsDNA after introduction into a cell (e.g., insertion of a virus into a host genome as a provirus). The expression construct may include one or more regions from a mammalian gene expressed in the microvasculature, especially endothelial cells (e.g., ICAM-2, tie), or a virus (e.g., adenovirus, adeno-associated virus, cytomegalovirus, herpes simplex virus, SV40 virus), as well as regions suitable for gene manipulation (e.g., selectable marker, linker with multiple recognition sites for restriction endonucleases, promoter for in vitro transcription, primer annealing sites for in vitro replication). The expression construct may be associated with proteins and other nucleic acids in a carrier (e.g., packaged in a viral particle or encapsulated in a liposome).

The expression construct further comprises a regulatory region for gene expression (e.g., promoter, enhancer, silencer, splice donor and acceptor sites, polyadenylation signal, cellular localization sequence). Transcription can be regulated by tetracycline or dimerized macrolides. The expression construct may be further comprised of one or more splice donor and acceptor sites within an expressed region; a Kozak consensus sequence upstream of an expressed region for initiation of translation; downstream of an expressed region, multiple stop codons in the three forward reading frames to ensure termination of translation, one or more mRNA degradation signals, a termination of transcription signal, a polyadenylation signal, and a 3′ cleavage signal. For expressed regions that do not contain an intron (e.g., a coding region from a cDNA), a pair of splice donor and acceptor sites may or may not be preferred. It would be useful, however, to include a mRNA degradation signal if it is desired to express one or more of the downstream regions only under the inducing condition. An origin of replication may be included that allows replication of the expression construct integrated in the host genome or as an autonomously replicating episome. Centromere and telomere sequences can also be included for the purposes of chromosomal segregation and protecting chromosomal ends from shortening, respectively. Random or targeted integration into the host genome is more likely to ensure maintenance of the expression construct but episomes could be maintained by selective pressure or, alternatively, may be preferred for those applications in which the expression construct is present only transiently.

An expressed region may be derived from a gene encoding LRP2 or its ligand apoJ in operative linkage with a regulatory region (e.g., constitutive, regulated, or endothelial-specific promoter and an optional enhancer). The expressed region may encode a translational fusion. Open reading frames of regions encoding a polypeptide and at least one heterologous domain may be ligated in register. If a reporter or selectable marker is used as the heterologous domain, then expression of the fusion protein may be readily assayed or localized. The heterologous domain may be an affinity or epitope tag.

### Screening of Candidate Compounds

Another aspect of the invention are chemical or genetic compounds, derivatives thereof, and compositions including same that are effective in the treatment of Alzheimer's disease and subjects at risk thereof. The amount that is administered to a subject in need of therapy or prevention, formulation, and timing and route of delivery is effective to reduce the number or severity of symptoms, to slow or limit progression of symptoms, to inhibit expression of one or more genes that are transcribed at a higher level in Alzheimer's disease, to activate expression of one or more genes that are transcribed at a lower level in Alzheimer's disease, or any combination thereof. Determination of such amounts, formulations, and timing and route of drug delivery is within the skill of persons conducting in vitro assays, in vivo studies of animal models, and human clinical trials.

A screening method may comprise administering a candidate compound to an organism or incubating a candidate compound with a cell, and then determining whether or not gene expression is increased. The increase in activity may partially or fully compensate for a change that is associated with or may cause Alzheimer's disease. Gene expression may be increased at the level of rate of transcriptional initiation, rate of transcriptional elongation, stability of the transcript, translation of the transcript, rate of translational initiation, rate of translational elongation, stability of protein, rate of protein folding, proportion of protein in active conformation, functional efficiency of protein (e.g., activation or repression of transcription), or combinations thereof. See U.S. Pat. Nos. 5,071,773 and 5,262,300. High-throughput screening assays are possible.

The screening method may comprise incubating a candidate compound with a cell containing a reporter construct, the reporter construct comprising transcription regulatory region covalently linked in a cis configuration to a downstream gene encoding an assayable product; and measuring production of the assayable product. A candidate compound which increases production of the assayable product would be identified as an agent which activates gene expression. See U.S. Pat. Nos. 5,849,493 and 5,863,733.

The screening method may comprise measuring in vitro transcription from a reporter construct in the presence or absence of a candidate compound, the reporter construct comprising a transcription regulatory region; and determining whether transcription is altered by the presence of the candidate compound. In vitro transcription may be assayed using a cell-free extract, partially purified fractions of the cell-free extract, purified transcription factors or RNA polymerase, or combinations thereof. See U.S. Pat. Nos. 5,453,362; 5,534,410; 5,563,036; 5,637,686; 5,708,158; and 5,710,025.

Techniques for measuring transcriptional or translational activity in vivo are known in the art. For example, a nuclear run-on assay may be employed to measure transcription of a reporter gene. Translation of the reporter gene may be measured by determining the activity of the translation product. The activity of a reporter gene can be measured by determining one or more of the abundance of transcription of polynucleotide product (e.g., RT-PCR of GFP transcripts), translation of polypeptide product (e.g., immunoassay of GFP protein), and enzymatic activity of the reporter protein per se (e.g., fluorescence of GFP or energy transfer thereof).

Gene activation may be achieved by inducing an expression construct containing a downstream region related to a gene that is down regulated (e.g., the full-length coding region or functional portions of the gene; hypermorphic mutants, homologs, orthologs, or paralogs thereof) or unrelated to the gene that acts to relieve suppression of gene activation (e.g., at least partially inhibiting expression of a negative regulator of the gene). Overexpression of transcription or translation, as well as overexpressing protein function, is a more direct approach to gene activation. Alternatively, the downstream expressed region may direct homologous recombination into a locus in the genome and thereby replace an endogenous transcriptional regulatory region of the gene with an expression cassette. In particular, gene expression of components of the receptor-ligand system transporting Aβ across the blood-brain barrier can be increased by introduction of an exogenous gene or activating an endogenous gene.

An expression construct may be introduced into a host cell (i.e., animal or human) or transgenic nonhuman mammal by a transfection or transgenesis technique using, for example, chemicals (e.g., calcium phosphate, DEAE-dextran, lipids, polymers), biolistics, electroporation, naked DNA technology, microinjection, or viral infection. The introduced expression construct may integrate into the host genome of the host cell or transgenic nonhuman mammal. Many neutral and charged lipids, sterols, and other phospholipids to make lipid carriers are known. For example, neutral lipids are dioleoylphosphatidylcholine (DOPC) and dioleoylphosphatidyl ethanolamine (DOPE); an anionic lipid is dioleoylphosphatidyl serine (DOPS); cationic lipids are dioleoyltrimethyl ammonium propane (DOTAP), dioctadecyldiamidoglycyl spermine (DOGS), dioleoyltrimethyl ammonium (DOTMA), and 1,3-dioleoyl-oxy-2-(6-carboxy-spermyl)-propylamide tetraacetate (DOSPER). Dipalmitoylphosphatidylcholine (DPPC) can be incorporated to improve the efficacy and/or stability of delivery. FUGENE 6, LIPOFECTAMINE, LIPOFECTIN, DMRIE-C, TRANSFECTAM, CELLFECTIN, PFX-1, PFX-2, PFX-3, PFX-4, PFX-5, PFX-6, PFX-7, PFX-8, TRANSFAST, TFX-10, TFX-20, TFX-50, and LIPOTAXI lipids are proprietary formulations. The polymer may be poly(β-amino esters), β-cyclodextrins, polyethylene glycol (PEG) or polyethylenimine (PEI); alternatively, polymeric materials can be formed into nanospheres or microspheres. Naked DNA technology delivers the expression construct in plasmid form to a cell, where the plasmid may or may not become integrated into the host genome, without using chemical transfecting agents (e.g., lipids, polymers) to condense the expression construct prior to introduction into the cell.

Thus, a host cell may be transfected with an expression construct; also provided are transgenic nonhuman mammals. In the previously discussed alternative, a homologous region from a gene can be used to direct integration to a particular genetic locus in the host genome and thereby regulate expression of the gene at that locus. Polypeptide may be produced in vitro by culturing transfected host cells; in vivo by transgenesis; or ex vivo by introducing the expression construct into allogeneic, autologous, histocompatible, or xenogeneic cells and then transplanting the transfected cells into a host organism. Special harvesting and culturing protocols will be needed for transfection and subsequent transplantation of host stem cells into a host mammal. Immunosuppression of the host mammal post-transplant or encapsulation of the host cells may be necessary to prevent rejection.

The expression construct may be used to replace the function of a gene that is down regulated or totally defective or supplement function of a partially defective gene. Thus, the cognate gene of the host may be neomorphic, hypomorphic, hypermorphic, or normal. Replacement or supplementation of function can be accomplished by the methods discussed above, and transfected mammalian cells or transgenic nonhuman mammals may be selected for high expression (e.g., assessing amount of transcribed or translated product, or physiological function of either product) of the downstream region. An animal model may be made by decreasing LRP2 activity in a nonhuman mammal by mutagenesis (e.g., promoter mutation that decreases transcription of a mouse Lrp2 gene, or missense mutation in Lrp2 coding region that decreases surface expression or receptor activity) or homologous recombination (e.g., deletion of a mouse Lrp2 gene). A normal phenotype in a subject (e.g., animal model, human) may be rescued by an expression construct that encodes at least a domain of LRP2 which binds the apoJ-Aβ complex and removes it from the CNS.

### Formulation of Compositions

Compounds of the invention or derivatives thereof may be used as a medicament or used to formulate a pharmaceutical composition with one or more of the utilities disclosed herein. They may be administered in vitro to cells in culture, in vivo to cells in the body, or ex vivo to cells outside of the subject that may later be returned to the body of the same subject or another. Such cells may be desegregated or provided as solid tissue.

Compounds or derivatives thereof may be used to produce a medicament or other pharmaceutical compositions. Use of compositions which further comprise a pharmaceutically acceptable carrier and compositions which further comprise components useful for delivering the composition to a subject are known in the art. Addition of such carriers and other components to the composition of the invention is well within the level of skill in this art.

Pharmaceutical compositions may be administered as a formulation adapted for passage through the blood-brain barrier or direct contact with the endothelium. Alternatively, pharmaceutical compositions may be added to the culture medium. In addition to the active compound, such compositions may contain pharmaceutically-acceptable carriers and other ingredients known to facilitate administration and/or enhance uptake (e.g., saline, dimethyl sulfoxide, lipid, polymer, affinity-based cell specific-targeting systems). The composition may be incorporated in a gel, sponge, or other permeable matrix (e.g., formed as pellets or a disk) and placed in proximity to the endothelium for sustained, local release. The composition may be administered in a single dose or in multiple doses which are administered at different times.

Pharmaceutical compositions may be administered by any known route. By way of example, the composition may be administered by a mucosal, pulmonary, topical, or other localized or systemic route (e.g., enteral and parenteral). The term “parenteral” includes subcutaneous, intradermal, intramuscular, intravenous, intra-arterial, intrathecal, and other injection or infusion techniques, without limitation.

Suitable choices in amounts and timing of doses, formulation, and routes of administration can be made with the goals of achieving a favorable response in the subject with Alzheimer's disease or at risk thereof (i.e., efficacy), and avoiding undue toxicity or other harm thereto (i.e., safety). Therefore, “effective” refers to such choices that involve routine manipulation of conditions to achieve a desired effect.

A bolus administered over a short time once a day is a convenient dosing schedule. Alternatively, the effective daily dose may be divided into multiple doses for purposes of administration, for example, two to twelve doses per day. Dosage levels of active ingredients in a pharmaceutical composition can also be varied so as to achieve a transient or sustained concentration of the compound or derivative thereof in a subject, especially in and around vascular endothelium of the brain, and to result in the desired therapeutic response or protection. But it is also within the skill of the art to start doses at levels lower than required to achieve the desired therapeutic effect and to gradually increase the dosage until the desired effect is achieved.

The amount of compound administered is dependent upon factors known to a person skilled in the art such as bioactivity and bioavailability of the compound (e.g., half-life in the body, stability, and metabolism); chemical properties of the compound (e.g., molecular weight, hydrophobicity, and solubility); route and scheduling of administration; and the like. For systemic administration, passage of the compound or its metabolite through the blood-brain barrier is important. It will also be understood that the specific dose level to be achieved for any particular subject may depend on a variety of factors, including age, gender, health, medical history, weight, combination with one or more other drugs, and severity of disease.

The term “treatment” of Alzheimer's disease refers to, inter alia, reducing or alleviating one or more symptoms in a subject, preventing one or more symptoms from worsening or progressing, promoting recovery or improving prognosis, and/or preventing disease in a subject who is free therefrom as well as slowing or reducing progression of existing disease. For a given subject, improvement in a symptom, its worsening, regression, or progression may be determined by an objective or subjective measure. Efficacy of treatment may be measured as an improvement in morbidity or mortality (e.g., lengthening of survival curve for a selected population). Preventative methods (e.g., preventing development of disease or the incidence of relapse) are also considered treatment. Reducing the systemic concentration of Aβ with antibody depletion or filtration of antibody-Aβ complexes may favor transport across the blood-brain barrier. Vasodilation, angiogenesis, neovascularization, and osmotic shock may be used to increase blood flow and thereby increase removal of Aβ from the brain to the systemic circulation. Another method for increasing removal may be to increase permeability of the blood vessel with known drugs (e.g., brakykinin, histamine); tight junctions may be loosened or the width increased to increase permeability. Other methods for increasing transcytosis and recycling of LRP2 may also be used (e.g., activators of cAMP signaling like theophylline). Treatment may also involve combination with other modes of treatment (e.g., ARICEPT or donepezil, EXELON or rivastigmine, anti-amyloid vaccine, mental exercise or stimulation, statins). Thus, combination treatment with one or more other drugs and one or more other medical procedures may be practiced.

The amount which is administered to a subject is preferably an amount that does not induce toxic effects which outweigh the advantages which result from its administration. Further objectives are to reduce in number, diminish in severity, and/or otherwise relieve suffering from the symptoms of the disease as compared to recognized standards of care.

Production of compounds according to present regulations will be regulated for good laboratory practices (GLP) and good manufacturing practices (GMP) by governmental agencies (e.g., U.S. Food and Drug Administration). This requires accurate and complete recordkeeping, as well as monitoring of QA/QC. Oversight of patient protocols by agencies and institutional panels is also envisioned to ensure that informed consent is obtained; safety, bioactivity, appropriate dosage, and efficacy of products are studied in phases; results are statistically significant; and ethical guidelines are followed. Similar oversight of protocols using animal models, as well as the use of toxic chemicals, and compliance with regulations is required.

### Methods

The brain clearance technique was used to determine Aβ clearance across the BBB and from CSF, as previously described (Shibata et al., 2000; Deane et al., 2004). Briefly, 0.5 μl of ¹²⁵I-Aβ42 (40 nM), ¹²⁵I-apoJ (40 nM) or ¹²⁵I-Aβ-apoJ (40 nM) was microinjected into the caudate nucleus simultaneously with ¹⁴C-inulin (an inert reference molecule as an index of interstitial fluid (ISF) bulk flow) and, after 30 minutes (a typical time point unless stated), the brain was removed and radiolabel was counted.

¹²⁵I-Aβ-apoJ complex was prepared by incubating ¹²⁵I-Aβ42 and apoJ at a molar ratio of 15 to 1 (¹²⁵I-Aβ42: apoJ) at 37° C. for 3 hours, and excess ¹²⁵I-Aβ42 was removed by ultrafiltration with a 30 kD molecular weight cut-off membrane.

In the CSF clearance studies, 0.5 μl of ¹²⁵I-apoJ (40 nM) was micro-injected into the right lateral ventricles (coordinates (mm) from bregma: antero-posterior −0.5, mediolateral +1.0, dorsoventral −2.2) simultaneously with ¹⁴C-inulin (the inert reference molecule), the brain was removed after 30 minutes, and radiolabel was counted.

### Calculations

Percentage ¹²⁵I-(TCA precipitable) recovery in brain=N_{B}/N_{I}×100. Where N_{B} is the ¹²⁵I-(TCA precipitable) recovered in brain and N_{I} is the ¹²⁵I-(TCA precipitable) injected into brain ISF or CSF. Total ¹²⁵I-(TCA precipitable) clearance from brain ISF consists of two components: clearance across the BBB and clearance via bulk flow of ISF. Therefore, clearance across the BBB was calculated from the difference between total ¹²⁵I-(TCA precipitable) clearance and ¹⁴C-inulin clearance (ISF bulk flow) as described (Shibata et al., 2000). Similarly, the percentage ¹²⁵I-(TCA precipitable) recovery in brain after microinjection into the CSF=N_{B}/N_{I}×100.

### Results

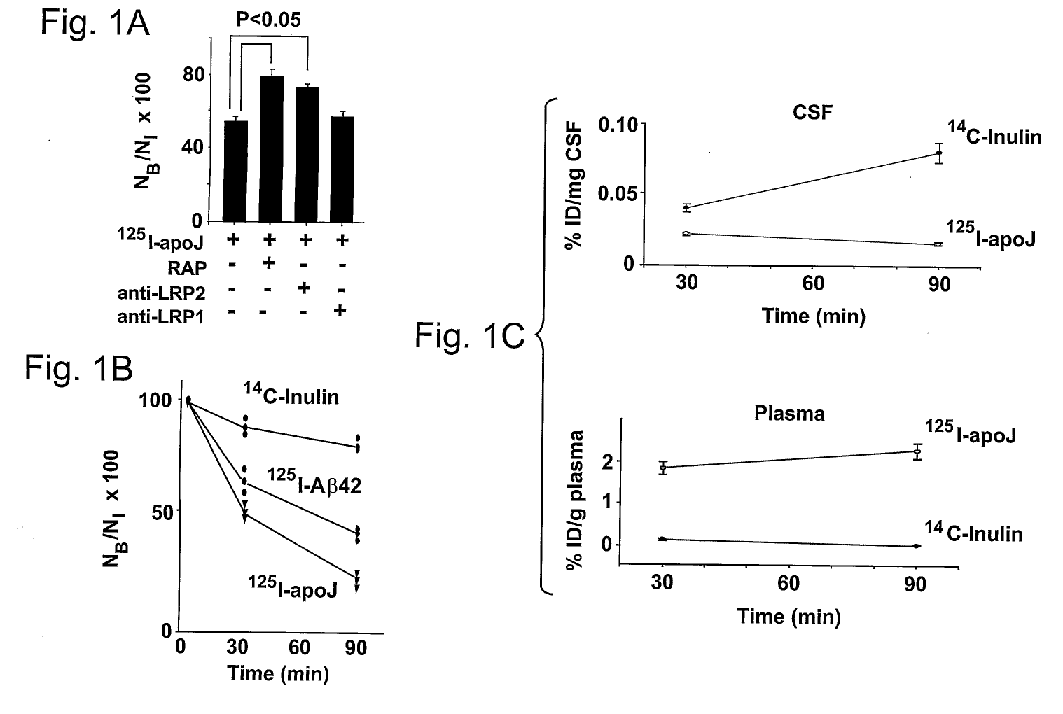

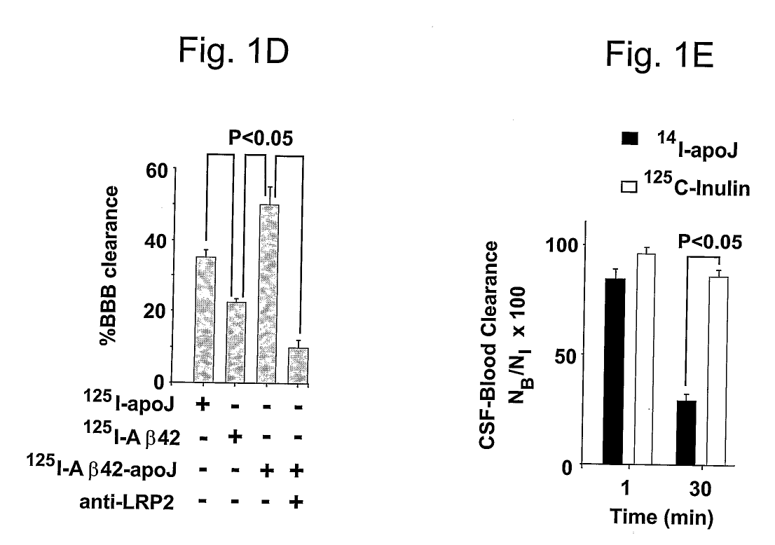

At physiological levels, 125I-apoJ (40 nM) is rapidly cleared from the brain ISF and this clearance is significantly reduced by RAP (receptor-associated protein, 5 μM) and anti-LRP2 (Rb 6286, from Dr. S. Argraves, 60 μg/ml), but not by anti-LRP1 (N20, Santa Cruz Biotech Inc., 60 μg/ml) (FIG. 1A). Its clearance from brain was substantially faster than that of ¹²⁵I-Aβ42 (40 nM) (FIG. 1B). ¹⁴C-inulin, an inert non-transported molecule, was slowly removed brain via bulk flow of brain ISF (FIG. 1B). Following microinjections of ¹²⁵I-apoJ and ¹⁴C-inulin into the brain, the time dependent appearance curves of TCA precipitable ¹²⁵I-apoJ radioactivity in CSF was decreased compared to ¹⁴C-inulin, while in plasma its levels were relatively increased compared to inulin (FIG. 1C). Clearance of ¹²⁵I-Aβ42 across the BBB from ¹²⁵I-Aβ42-apoJ (40 nM) complexes was significantly higher compared to clearance of free uncomplexed monomeric ¹²⁵I-Aβ42 across the BBB, i.e., from 22%±2% ID to 48%±4% (n=3, P<0.05). Anti-LRP2 antibody reduced the ¹²⁵I-Aβ42-apoJ BBB clearance to 12%±2% (n=3, P<0.05) (FIG. 1D). There was no a significant change in ¹²⁵I-β42 clearance via the ISF bulk flow (not shown). ¹²⁵I-apoJ (40 nM) was rapidly cleared from the CSF as compared to ¹⁴C-inulin, after 30 minutes, following microinjection of ¹²⁵I-apoJ (40 nM) and ¹⁴C-inulin into the lateral ventricles (FIG. 1E).

### CONCLUSIONS

LRP2, the apoJ receptor present on the CNS vascular barriers (BBB and choroid plexus) rapidly clears apoJ at the physiological concentrations from brain and CSF. It also facilitates elimination of Aβ42, an amyloidogenic Aβ peptide with high β-sheet content, which requires apoJ-assisted clearance. Thus, strategies which enhance LRP2 expression at the vascular CNS barriers hold potential of enhancing clearance of high β-sheet content Aβ species from the CNS via apoJ.

### REFERENCES

Boggs et al. (1996) Clusterin (Apo J) protects against in vitro amyloid-β(1-40) neurotoxicity. J. Neurochem. 67, 1324-1327
Choi-Miura et al. (1992) SP-40,40 is a constituent of Alzheimer's amyloid. Acta Neuropath. 83, 260-264
Chun et al. (1999) Glycoprotein 330/megalin (LRP-2) has low prevalence as mRNA and protein in brain microvessels and choroid plexus. Exp. Neurol. 157,
Danik et al. (1993) Localization of sulfated glycoprotein-2/clusterin mRNA in the rat brain by in situ hybridization. J. Comp. Neurol. 334, 209-227
Deane et al. (2004) LRP/amyloid β-peptide interaction mediates differential brain efflux of Aβ isoforms. Neuron 43, 333-344
DeMattos et al. (2002) Clusterin promotes amyloid plaque formation and is critical for neuritis toxicity in a mouse model of Alzheimer's disease. Proc. Natl. Acad. Sci. USA 99, 10843-10848
Fagan et al. (1999) Unique lipoproteins secreted by primary astrocytes from wild type, apoE−/− and human apoE transgenic mice. J. Biol. Chem. 274,
Ghiso et al. (1993) The cerebrospinal-fluid soluble form of Alzheimer's amyloid beta is complexed to SP-40,40 (apolipoprotein J), an inhibitor of the complement membrane-attack complex. Biochem. J. 293, 27-30
Jenne & Tschopp (1992) Clusterin: The intriguing guises of a widely expressed glycoprotein. Trends Biochem. Sci. 17, 154-159
Koudinov et al. (1994) The soluble form of Alzheimer's amyloid P protein is complexed to high density lipoprotein 3 and very high density lipoprotein in normal human plasma. Biochem. Biophys. Res. Commun. 205, 1164-1171
Kounnas et al. (1995) Identification of glycoprotein 330 as an endocytic receptor for apolipoprotein J/clusterin. J. Biol. Chem. 270, 13070-13075
Lambert et al. (1998) Diffusible, nonfibrillar ligands derived from Aβ1-42 are potent central nervous system neurotoxins. Proc. Natl. Acad. Sci. USA 95,
McGeer et al. (1992) Distribution of clusterin in Alzheimer brain tissue. Brain Res. 579, 337-341
Matsubara et al. (1996) Apolipoprotein J and Alzheimer's amyloid β solubility. Biochem. J. 316, 671-679
May. & Finch (1992) Sulphated glycoprotein-2: New relationship of this multifunctional protein to neurodegeneration. Trends Neurosci. 15, 391-396
May et al. (1989) Altered gene expression in Alzheimer's disease brain tissue. Can. J. Neurol. Sci. 16, 473-476
Oda et al. (1995) Clusterin (apoJ) alters the aggregation of amyloid β-peptide (Aβ1-42) and forms slowly sedimenting Aβ complexes that cause oxidative stress. Exp. Neurol. 136, 22-31
Pasinetti et al. (1993) Astrocytic responses to striatal differentiation in male rat. Neuroscience 53, 199-211
Rebeck & Hyman (1999) Lipoprotein receptors in brain In: Clusterin in Normal Brain Function & During Neurodegeneration (ed. Finch, C.) pp. 49-59 (R. G. Landes, Austin, Tex.)
Shibata et al. (2000) Clearance of Alzheimer's amyloid-β 1-40 peptide from brain by LDL receptor-related protein-1 at the blood-brain barrier. J. Clin. Invest. 106, 1489-1499
Zlokovic et al. (1996) Glycoprotein 330/megalin: Probable role in receptor-mediated transport of apolipoprotein J alone and in a complex with Alzheimer's disease amyloid-β at the blood-brain and blood-cerebrospinal fluid barriers. Proc. Natl. Acad. Sci. USA 93, 4229-4234

Patents, patent applications, books and other publications cited herein are incorporated by reference in their entirety.

All modifications and substitutions that come within the meaning of the claims and the range of their legal equivalents are to be embraced within their scope. A claim which recites “comprising” allows the inclusion of other elements to be within the scope of the claim; the invention is also described by such claims reciting “consisting essentially of” (i.e., allowing the inclusion of other elements to be within the scope of the claim if they do not materially affect operation of the invention) or “consisting of” (i.e., allowing only the elements listed in the claim other than impurities or inconsequential activities which are ordinarily associated with the invention) instead of the “comprising” term. Any of these three transitions can be used to claim the invention.

It should be understood that an element described in this specification should not be construed as a limitation of the claimed invention unless it is explicitly recited in the claims. Thus, the granted claims are the basis for determining the scope of legal protection instead of a limitation from the specification which is read into the claims. In contradistinction, the prior art is explicitly excluded from the invention to the extent of specific embodiments that would anticipate the claimed invention or destroy novelty.

Moreover, no particular relationship between or among limitations of a claim is intended unless such relationship is explicitly recited in the claim (e.g., the arrangement of components in a product claim or order of steps in a method claim is not a limitation of the claim unless explicitly stated to be so). All possible combinations and permutations of individual elements disclosed herein are considered to be aspects of the invention. Similarly, generalizations of the invention's description are considered to be part of the invention.

From the foregoing, it would be apparent to a person of skill in this art that the invention can be embodied in other specific forms without departing from its spirit or essential characteristics. The described embodiments should be considered only as illustrative, not restrictive, because the scope of the legal protection provided for the invention will be indicated by the appended claims rather than by this specification.

1. A method of at least diagnosing a subject with Alzheimer's disease or identifying a subject as being at risk for developing Alzheimer's disease, said method comprising:
(a) measuring at least abundance of low-density lipoprotein receptor-related protein 2 (LRP2), abundance of transcripts thereof, or LRP2 receptor activity in a subject;
(b) comparing the abundance of LRP2 protein, abundance of transcripts thereof, or activity of LRP2 receptor in the subject to at least an age-matched control not affected by Alzheimer's disease; and
(c) at least diagnosing the subject with Alzheimer's disease or identifying the subject as being at risk for developing Alzheimer's disease when the abundance of LRP2 protein, abundance of transcripts thereof, or activity of LRP2 receptor in the subject is decreased relative to said age-matched control not affected by Alzheimer's disease.

2. The method of claim 1, wherein the abundance of LRP2 protein, abundance of transcripts thereof, or activity of LRP2 receptor is measured at least in a brain vascular or systemic endothelial cell of the subject.

3. The method of claim 1, wherein the abundance of LRP2 protein, abundance of transcripts thereof, or activity of LRP2 receptor is measured at least in a brain capillary, a temporal artery, a leptomeningeal artery, or at the blood-brain barrier of the subject.

4. A method of at least treating a subject with Alzheimer's disease or at risk for developing Alzheimer's disease, said method comprising increasing low-density lipoprotein receptor-related protein 2 (LRP2)-mediated removal of amyloid-β peptide (Aβ) at the blood-brain barrier in the subject in the direction from brain to blood or increasing low-density lipoprotein receptor-related protein 2 (LRP2)-mediated clearance of amyloid-β peptide (Aβ) from the central nervous system of the subject.

5. The method of claim 4, wherein removal of Aβ is increased by increasing at least LRP2 expression or receptor activity.

6. The method of claim 4, wherein removal of Aβ is increased by increasing at least expression of apolipoprotein J (apoJ) or transport of apoJ.

7. The method of claim 4, wherein low-density lipoprotein receptor-related protein 2 (LRP2)-mediated clearance of amyloid-β peptide (Aβ) from the central nervous system of the subject is increased.

8. The method of claim 4, wherein an expression construct encoding at least a domain of low-density lipoprotein receptor-related protein 2 (LRP2) which binds a complex of apolipoprotein J (apoJ) and amyloid-β peptide (Aβ) is used to treat Alzheimer's disease.

9. A method of determining the effectiveness of at least a drug or medical regimen for treating Alzheimer's disease, said method comprising:
(a) measuring the abundance of low-density lipoprotein receptor-related protein 2 (LRP2), abundance of transcripts thereof, or activity of LRP2 receptor in the presence of said drug or medical regimen, and
(b) at least determining said drug or medical regimen is effective when the abundance of LRP2 protein, abundance of transcripts thereof, or activity of LRP2 receptor is increased in the presence of said drug or medical regimen.

10. A novel drug or use of a medical regimen for treating Alzheimer's disease determined to be effective by the method of claim 9.

11. A kit for use in the method of claim 1, the kit comprised of one or more of a specific binding molecule for low-density lipoprotein receptor-related protein 2 (LRP2) or a transcript thereof used to measure abundance of said LRP2 protein, abundance of transcripts thereof, or LRP2 receptor activity and, optionally, a non-Alzheimer's disease control containing LRP2 protein or a transcript thereof.
